(19) United States
(12) Patent Application Publication
(10) Pub. No.: US 2006/0128628 A1
(43) Pub. Date: Jun. 15, 2006

(54) Human tissue antigen-binding peptides and their amino acid sequences

(76) Inventors: Show-Li Chen, Taipei (TW); Ching-Jen Yang, Taipei (TW); Yeou-Ping Tsao, Taipei (TW)

Correspondence Address: RABIN & BERDO, P.C., Suite 500, 1101 14 Street, Washington, DC, 20005, US

(21) Appl. No.: 11/011,026

(22) Filed: Dec. 15, 2004

(51) Int. Cl. A61K 38/10; C07K 7/08

(52) U.S. Cl. 514/15; 530/328

(57) ABSTRACT

The invention relates to a peptide that has epitopes with amino acid sequences selected from the S protein of SARS virus. Such peptides can bind human tissue antigen to form a complex, and can induce cytotoxicity. The invention relates further to a vaccine or diagnostic agent formed by combining said peptides with suitable carrier, adjuvant or diluent.

### BACKGROUND OF THE INVENTION

1. Field of the invention

The invention relates to peptides and their amino acid sequences, and in particular, to peptides derived from viruses, being able to bind to human tissue antigens and capable of inducing immune response.

2. Description of the prior art

Atypical pneumonia has been burst and spread over the whole world and, as such, this syndrome has been denomiated as Severe Acute Respiratory Syndrome (SARS). Up to now, SARS has been identified as been caused by Coronavirus family and denomiated as Urbani SARS-associated coronavirus. RNA of Coronavirus is capped on its one end, and polyadenylated on another end in such a way that RNA of Coronavirus can be functioned and infectious like an mRNA. Coronavirus has a leader RNA with length of 60-80 nucleotides at the 5′ end, and followed with a sequence of 200-500 nucleotides. The size of the first 60% length of sequence from the 5′ end is approximately 20 kb. This sequence comprises two overlapping open reading frame (ORF), ORF1a and ORF1b, and encodes RNA polymerase, protease, and other proteins with unidentified characteristics.

In the overlapping region between ORF1a and ORF1b, there is an unidentified sequence of 7 nucleotides, and a pseudoknot that is essentially a frameshift of a ribosome. One member of the Coronavirus family is Infectious Bronchitis Virus (IBV) that can cause highly infectious respiratory diseases among fowls. Therefore, fowls can be used as animal models of immune research for studying the mechanism involved in virus control. Cytotoxicity is essential for controlling the early infection of IBV. Therefore, to define the epitope of the peptide on S protein which binding to human tissue antigen 0201 to produce cytotoxicity is the major approach of vaccine development in the future.

US Pub. No.: 20030103946 (hereafter the cited reference) provided a peptide which can bind to HLA and is derived from the human CD45 polypeptide or part or derivatives thereof, and hence is an incomplete human CD polypeptide. Preferably, said peptide comprises following amino acid sequence: FLYDVIAST, ALIAFLAFL, KLFTAKLNV, MIWEQKATV, NLSELHPYL, VNLSELHPY, LLAFGFAFL, YLYNKETKL, LILDVPPGV, TLILDVPPGV, ILYNNHKFT, ILPYDYNRV, YILIHQALV, FQLHDCTQV, KLLAFGFAFL, YQYQYTNWSV or part or derivative thereof. Further, the cited reference revealed a method for treating leukemia using specific T lymphocyte immunotherapy.

However, there are few patents or literatures relating to peptides that derived from other pathogens and are able to bind to human tissue antigen, and hence much space remains to be developed. In view of this, the inventors, after compiling for many years and carrying out extensively experiments designed in-depth, have identified finally a peptide from the amino acid sequence of S protein of SARS virus, characterized in that this peptide can bind to the human tissue antigen and hence induce the immune response.

### SUMMARY OF THE INVENTUON

Accordingly, one object of the invention is to provide a peptide that can bind to human tissue antigen 0201 and hence induce cytotoxicity, and in particular, to provide such a peptide having an amino acid sequence derived from SARS virus.

A peptide that can bind to human tissue antigen and that can accomplish the above-mentioned object of the invention comprises one of the following amino acid sequences: VVFLHVTYV or ILPDPLKPT and derivatives thereof. Such a peptide can be screened out of the S protein of SARS virus and can bind to a human tissue antigen 0201 (HLA-0201) to form a human major histocompatibility complex (MHC) and hence induce cell immune response, and consequently, it can be used to prepare vaccines and diagnostic agent.

These features and advantages of the present invention will be fully understood and appreciated from the following detailed description of the accompanying drawings.

### BRIEF DESCRIPTION OF THE DRAWINGS

FIG. 1 shows the result of T2 cell binding test;

FIG. 2A and B show results of expressing CD8+ and IFN-γ+ in transgenic mice after immunization with S2 peptide; and

FIG. 3A and B show results of expressing CD8+ and IFN-γ+ in transgenic mice after immunization with S6 peptide.

### DETAILED DESCRIPTION OF THE PREFERRED EMBODIMENT

### EXAMPLE 1

From the information relating human tissue antigen binding peptide provided by the National Institutes of Health of USA, peptides that could bind to human tissue antigen 0201 (HLA-0201) and that had a gene sequence homologous to the allele-specific motifs of human MHC-I molecule HLA-0201 were screened from S protein of SARS virus. Possible CTL epitopes were labeled by evaluating the dissociation time of a molecule containing 9 amino acids from MHC-1. After further screening peptides containing epitopes that exhibited cytotoxicity possibly, ten peptides (S1˜S10) that might contain amino acid sequences of S protein peptides and that could bind specifically to human tissue antigen 0201 were obtained (as shown in Table 1).

TABLE 1

| Peptide Name | Start Position | Peptide Sequence |
|---|---|---|
| S1 | 1174-1182 | NLNESLIDL |
| S2 | 1042-1050 | VVFLHVTYV |
| S3 | 982-990 | RLQSLQTYV |
| S4 | 958-966 | VLNDILSRL |
| S5 | 897-905 | VLYENQKQI |
| S6 | 787-795 | ILPDPLKPT |
| S7 | 734-742 | LLLQYGSFC |
| S8 | 411-419 | KLPDDFMGC |
| S9 | 151-159 | MIFDNAFNC |
| S10 | 2-10 | FIFLLFLTL |

These S protein peptides (S1˜S10) described above were then synthesized by an automatic peptide synthesizer (Abimed AMS 422), dissolved in 1 to 5 mg/ml dimethyl sulfoxide (DMSO), and stored at −70° C. in liquid form till analyzed by reverse phase high performance liquid chromatography.

### EXAMPLE 2

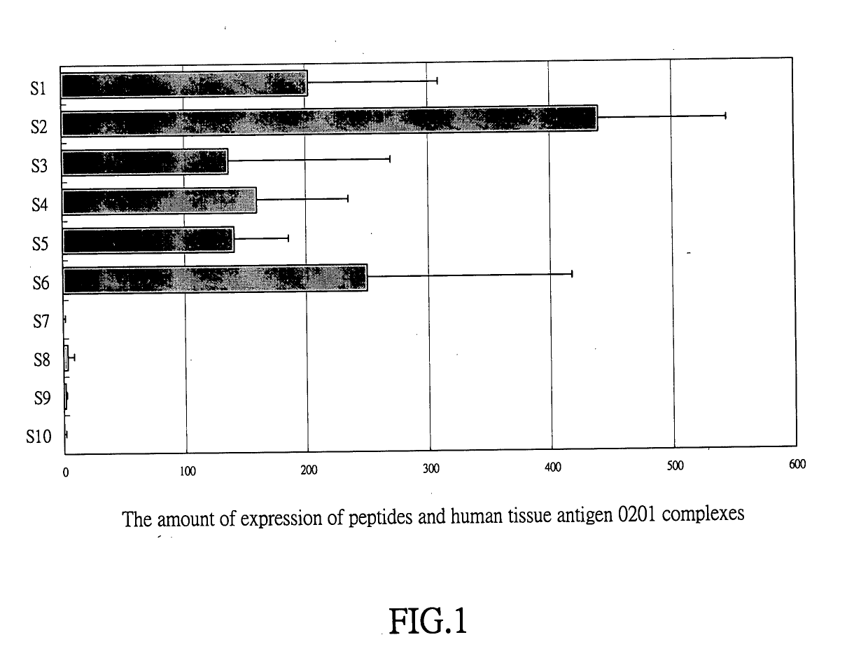

In this example, we verified those peptides that could bind to human tissue antigen 0201(HLA-A0201) by testing the binding of the above-mentioned peptides (S1˜S10) to T2 cell. T2 cell line is a cell line lacking of the transporters associated with antigen processing (TAP), TAP1 and TAP2, and can express small amount of human tissue antigen 0201. A T2 cell binding test was carried out at 37° C. by immobilizing HLA-0201 on the surface of T2 cell with peptide (S1˜S10) in order to screen peptides binding strongly to the human tissue antigen 0201. After binding, the major tissue antigen-peptide complexes were formed at the surface of the cell. The amount of human lymphocyte antigen A2 expressed and the median fluorescence intensity was calculated by flow cytometer. Results shown in FIG. 1 indicated that peptides S1, S2, S3, S4, S5 and S6 could bind to the human tissue antigen 0201.

### EXAMPLE 3

Thereafter, two peptides with strongest binding ability (S2 and S6) were determined whether they can induce antigen-specific cytotoxicity.

This experiment was carried out using transgenic mice with human tissue antigen 0201. These transgenic mice were C57BL/6 mice that could express human tissue antigen 0201. According to results of the T2 cell binding test, S2 and S6 were peptides that possessed most likely epitopes. Mice were injected intramuscularly every week with peptides and CpG ODN 1826 mixed vaccine. At the fifth day after the third vaccine injection, number of lymphocytes of the transgenic mice that can express simultaneously CD8+ and IFN-γ+ were measured by flow cytometer. Results indicated that peptides S2 and S6 could induce cytotoxicity.

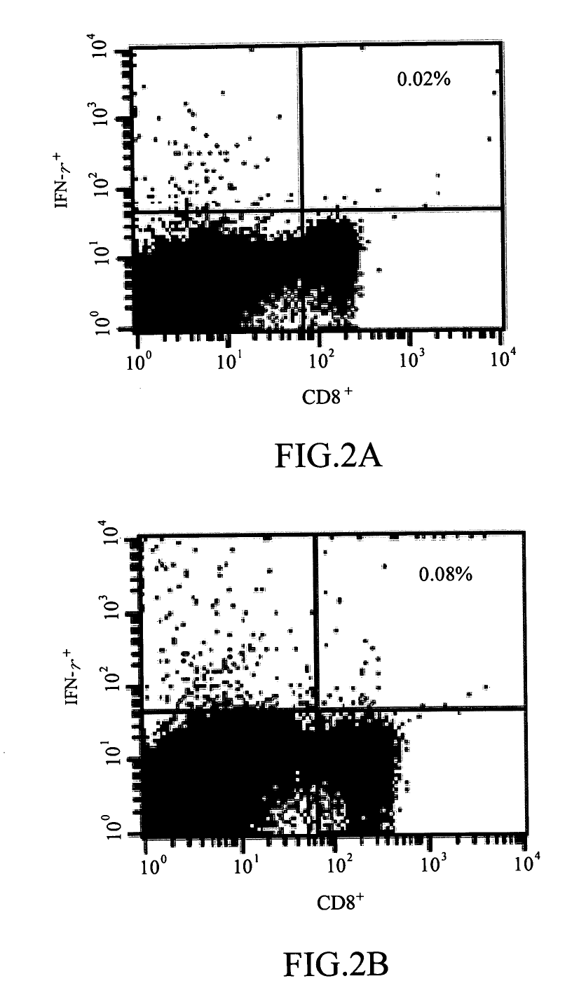

Spleen lymphocytes obtained from those peptide-immunized HLA-0201 -trangenic mice were allowed to bind with stimulants (S synthetic peptide) to detect the S peptide-specific CD8+ cell precursor. Before collecting these cells, the interferon (INF-γ+) produced by the spleen lymphocytes was accumulated within Golgi apparatus by using a reagent. After washing cells twice, the mouse anti-CD8 monoclonal antibody conjugated with FITC was used to detect the mouse antigen CD8b.2 on spleen lymphocytes. Spleen lymphocytes from transgenic mice were counted by flow cytometry and numbers of those lymphocytes that could express simultaneously CD8+ and IFN-γ+ were determined. FIG. 2A and C show the results of expressing CD8+ and IFN-γ+ by lymphocytes that had been stimulated with vaccine containing S2 peptide in amounts of 0 and 10 μg, respectively. Black spots within the box at right upper corner of each Figure indicated the proportion of spleen lymphocytes that could express simultaneously sufficient amounts of CD8+ and IFN-γ+ and that could induce cytotoxicity. Results revealed that vaccines containing S2 peptide could induce cytotoxicity.

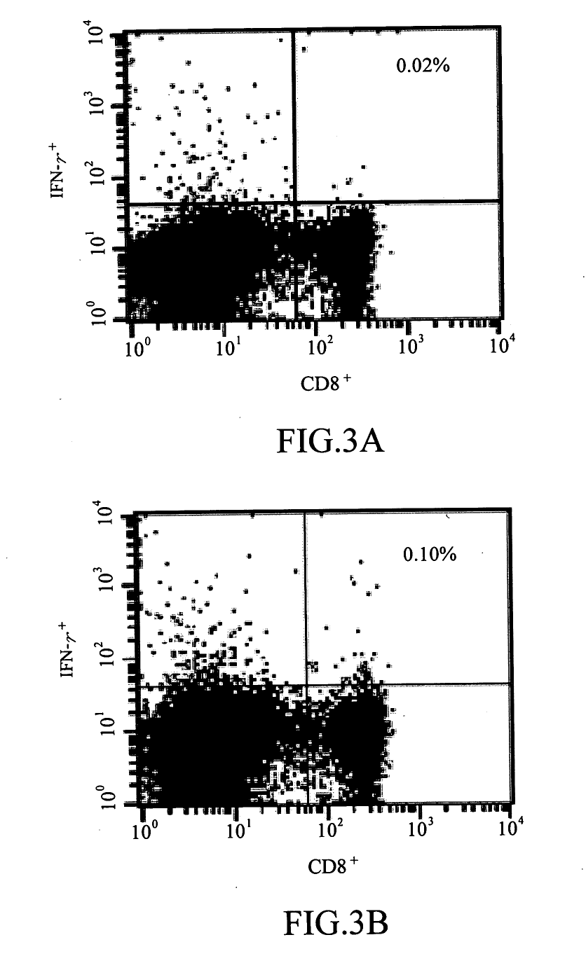

FIG. 3A and B show results of expressing CD8+ and IFN-γ+ by lymphocytes that had been stimulated with vaccine containing S6 peptide in amounts of 0 and 10 μg, respectively. As described above, black spots within the box at right upper corner of each Figure indicated the proportion of spleen lymphocytes that could express simultaneously sufficient amounts of CD8+ and IFN-γ+ and that could induce cytotoxicity. Results revealed that vaccines containing S6 peptide could induce cytotoxicity.

As compared with the technique disclosed in the cited reference, peptides provided according to the invention comprise further following characteristics and advantages:
1. Peptides described in the cited reference were derived from human CD45, whereas peptides disclosed according to the invention were derived from S protein of SARS virus.
2. The amino acid sequences of peptides described in the cited reference were different from those of the peptides disclosed according to the invention.
3. Peptides according to the invention can be used in the production of vaccines and diagnostic agents for preventing and treating the infection of SARS virus.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. A peptide capable of binding a human tissue antigen comprises at least an amino acid sequence selected from the group consisting of VVFLHVTYV (SEQ ID No: 2) and derivatives thereof.

2. A peptide capable of binding a human tissue antigen according to claim 1, wherein said human tissue antigen is human tissue antigen 0201.

3. A peptide capable of binding a human tissue antigen according to claim 1, wherein said amino acid sequence is a fragment of S protein from SARS virus or derivatives of said fragment.

4. A peptide capable of binding a human tissue antigen according to claim 1, wherein said derivatives are amino acid-substituted derivatives.

5. A peptide capable of binding a human tissue antigen according to claim 1, wherein said fragment contain epitopes and can bind human tissue antigen to induce cytotoxicity.

6. A peptide capable of binding a human tissue antigen comprises at least an amino acid sequence selected from fragments of S protein of SARS virus or derivatives of said fragments.

7. A peptide capable of binding a human tissue antigen according to claim 6, wherein said human tissue antigen is human tissue antigen 0201.

8. A peptide capable of binding a human tissue antigen according to claim 6, wherein said amino acid sequence is selected from the group consisting of VVFLHVTYV (SEQ ID No: 2) and derivatives thereof.

9. A peptide capable of binding a human tissue antigen according to claim 6, wherein said derivatives are amino acid-substituted derivatives.

10. A peptide capable of binding a human tissue antigen according to claim 8, wherein said derivatives are amino acid-substituted derivatives.

11. A peptide capable of binding a human tissue antigen according to claim 6, wherein said fragments contain epitopes and can bind human tissue antigen to induce cytotoxicity.

12. A peptide capable of binding a human tissue antigen according to claim 1, within said peptide could bind to a human tissue antigen to form a peptide complex.

13. A peptide capable of binding a human tissue antigen according to claim 6, within said peptide could bind to a human tissue antigen to become a peptide complex.

14. A peptide capable of binding a human tissue antigen according to claim 12, within said peptide complex comprises human tissue antigen 0201.

15. A peptide capable of binding a human tissue antigen according to claim 13, within said peptide complex comprises human tissue antigen 0201.

16. A peptide capable of binding a human tissue antigen according to claim 1, within said peptide could combine with pharmaceutically acceptable carrier, adjuvant or diluent to produce a vaccine.

17. A peptide capable of binding a human tissue antigen according to claim 6, within said peptide could combine with pharmaceutically acceptable carrier, adjuvant or diluent to produce a vaccine.

18. A peptide capable of binding a human tissue antigen according to claim 1, within said peptide could combine with pharmaceutically acceptable adjuvant or diluent to produce a detection reagent for SARS virus.

19. A peptide capable of binding a human tissue antigen according to claim 6, within said peptide could combine with pharmaceutically acceptable adjuvant or diluent to produce a diagnostic reagent for SARS virus.


SEQUENCE LISTING (26 sequences)

SEQ ID NO: 1 - protein, 9 aa, Homo sapiens
NLNESLIDL

SEQ ID NO: 2 - protein, 9 aa, Homo sapiens
VVFLHVTYV

SEQ ID NO: 3 - protein, 9 aa, Homo sapiens
RLQSLQTYV

SEQ ID NO: 4 - protein, 9 aa, Homo sapiens
VLNDILSRL

SEQ ID NO: 5 - protein, 9 aa, Homo sapiens
VLYENQKQI

SEQ ID NO: 6 - protein, 9 aa, Homo sapiens
ILPDPLKPT

SEQ ID NO: 7 - protein, 9 aa, Homo sapiens
LLLQYGSFC

SEQ ID NO: 8 - protein, 9 aa, Homo sapiens
KLPDDFMGC

SEQ ID NO: 9 - protein, 9 aa, Homo sapiens
MIFDNAFNC

SEQ ID NO: 10 - protein, 9 aa, Homo sapiens
FIFLLFLTL

SEQ ID NO: 11 - protein, 9 aa, Homo sapiens
FLYDVIAST

SEQ ID NO: 12 - protein, 9 aa, Homo sapiens
ALIAFLAFL

SEQ ID NO: 13 - protein, 9 aa, Homo sapiens
KLFTAKLNV

SEQ ID NO: 14 - protein, 9 aa, Homo sapiens
MIWEQKATV

SEQ ID NO: 15 - protein, 9 aa, Homo sapiens
NLSELHPYL

SEQ ID NO: 16 - protein, 9 aa, Homo sapiens
VNLSELHPY

SEQ ID NO: 17 - protein, 9 aa, Homo sapiens
LLAFGFAFL

SEQ ID NO: 18 - protein, 9 aa, Homo sapiens
YLYNKETKL

SEQ ID NO: 19 - protein, 9 aa, Homo sapiens
LILDVPPGV

SEQ ID NO: 20 - protein, 10 aa, Homo sapiens
TLILDVPPGV

SEQ ID NO: 21 - protein, 9 aa, Homo sapiens
ILYNNHKFT

SEQ ID NO: 22 - protein, 9 aa, Homo sapiens
ILPYDYNRV

SEQ ID NO: 23 - protein, 9 aa, Homo sapiens
YILIHQALV

SEQ ID NO: 24 - protein, 9 aa, Homo sapiens
FQLHDCTQV

SEQ ID NO: 25 - protein, 10 aa, Homo sapiens
KLLAFGFAFL

SEQ ID NO: 26 - protein, 10 aa, Homo sapiens
YQYQYTNWSV